(19) United States
(12) Patent Application Publication
(10) Pub. No.: US 2004/0087891 A1
(43) Pub. Date: May 6, 2004

(54) Liquid displacement arrangement

(76) Inventor: Pawel Wisniewski, Durban (SA)

Correspondence Address: J Rodman Steele Jr, Akeman Senterfitt & Eidson, PO Box 3188, West Palm Beach, FL, 33402-3188, US

(21) Appl. No.: 10/472,672

(22) PCT Filed: Mar. 22, 2002
(86) PCT No.: PCT/IB02/00850

(30) Foreign Application Priority Data
Mar. 26, 2001 (ZA) 2001/2455

(51) Int. Cl. A61M 5/178

(52) U.S. Cl. 604/36

(57) ABSTRACT

The invention relates to a liquid displacement arrangement for displacing a liquid in a controlled manner to the body of a person, such displacement being required particularly in relation to procedures associated with the manipulation or destruction of ureteric and kidney stones. The liquid displacement arrangement includes in combination a syringe-type pump, a feed arrangement for feeding liquid in a controlled unidirectional mode from a supply container to the body of a person and a foot operated operating mechanism on which the syringe-type pump can be releasably located for the operation thereof. Use of the liquid displacement arrangement permits the person performing a procedure to control himself the feed of liquid required for the procedure.

THIS INVENTION relates to a liquid displacement arrangement for displacing a liquid in a controlled manner to the body of a person.

It is required in relation to various medical procedures to displace a liquid in a controlled manner to the body of a person as a part of the procedures. This displacement of liquid is manually controlled by using a syringe-type liquid displacement pump, often by an assistant in response to instructions of the person performing the procedure. Although the person performing the procedure thus has both hands available to perform the procedure, to control the manual actions of an assistant through verbal instructions can be problematic and cause difficulties within procedures.

It is thus an object of this invention to at least alleviate the above problem.

Although reference is made herein particularly to the displacement of a liquid to the body of a person, procedures requiring similar displacement to the body of an animal also exist. As such, any reference herein to the displacement of a liquid to the body of a person must be interpreted as a reference also to the displacement of a liquid to the body of an animal. It must be understood also that references to displacement of a liquid to the body of a person must be interpreted to include references to the displacement of a liquid into the body of a person, typically via a liquid feed means such as a needle, or the like. Whether a liquid is to be displaced into the body of a person or to the body of a person clearly will be determined in practice by the procedure being performed.

According to the invention there is provided a liquid displacement arrangement for displacing a liquid in a controlled manner to the body of a person, which comprises

a syringe-type pump including a cylinder, a piston displaceable within the cylinder and a piston rod extending from the piston and projecting from the cylinder to enable displacement of the piston and thereby operation of the pump;

an inflow tube of which one end is connected in liquid communication with the cylinder and the opposite free end is connectable to a liquid supply, the inflow tube having a unidirectional valve located in line therewith that permits only flow towards the cylinder, during operation of the pump;

an outflow tube of which one end is connected in liquid communication with the cylinder and the opposite free end is connectable to a liquid feed means for feeding a liquid to the body of a person, the outflow tube having a unidirectional valve located in line therewith that permits only flow from the cylinder, during operation of the pump,

and in which the piston rod and the cylinder of the syringe-type pump each define a locating formation for removably locating the pump between a base member and a foot pedal of a foot operated operating mechanism in a configuration in which displacement of the foot pedal provides for required displacement of the piston of the pump for the operation of the pump.

The liquid displacement arrangement may include a connector formation which communicates with the cylinder of the syringe-type pump and to which the inflow tube and the outflow tube are connected for providing their required liquid communication with the cylinder.

The free end of the inflow tube of the liquid displacement arrangement may be connectable to a liquid supply container while permitting the container to be positioned at different height levels with respect to the body of a person to which a liquid from the liquid supply container must be displaced, in use of the liquid displacement arrangement, and, as such, the unidirectional valves located in line with the inflow tube and the outflow tube respectively, may permit liquid flow in the permitted direction when the hydrostatic pressure acting on the valves, being determined by the position of a liquid supply container to which the inflow tube is connected, in use, exceeds a predetermined pressure level.

The liquid displacement arrangement of the invention also may include a foot operated operating mechanism, for operating the syringe-type pump, as a part thereof, the operating mechanism comprising

a base member positionable on a floor surface; and

a foot pedal displaceably mounted on the base member,

the base member and the foot pedal each defining a locating formation complementary to one of the locating formations defined respectively by the piston rod and the cylinder of the syringe-type pump, permitting the removable location of the pump between the base member and the foot pedal in a configuration in which displacement of the foot pedal with respect to the base member provides for operation of the pump.

Particularly for use with a liquid displacement arrangement including such a foot operated operating mechanism, the locating formations defined respectively by the piston rod and the cylinder of the syringe-type pump may be located at the operative remote ends of the piston rod and the cylinder.

The foot pedal of the foot operated operating mechanism may be pivotally mounted on the base member and the locating formations may permit relative pivotal movement respectively between the syringe-type pump and the base member and the syringe-type pump and the foot pedal, the relative pivotal movement permitting axial displacement of the piston of the syringe-type pump by the displacement of the foot pedal.

Further according to the invention, the foot pedal may be displaceable between a retracted position and a depressed position, which positions respectively coincide substantially with limit positions of the piston of the syringe-type pump within its cylinder. As such, in use of the liquid displacement arrangement, displacement of the pedal from its retracted position to its depressed position may provide for displacement of liquid from the cylinder of the syringe-type pump, whereas displacement of the foot pedal from its depressed position to its retracted position may provide for displacement of liquid into the cylinder.

The foot operated operating mechanism also may include urging means acting between the base member and the foot pedal for urging the foot pedal into its retracted position. Still further, the foot operated foot operating mechanism may include a stop arrangement that acts between the foot pedal and the base member for determining the fully retracted position and the fully depressed position of the pedal with respect to the base member.

The syringe-type pump of the liquid displacement arrangement of the invention may include urging means acting on the piston rod for urging the piston rod into its fully extended position with respect to the cylinder.

A preferred embodiment of the liquid displacement arrangement of the invention provides for the syringe-type pump, the inflow tube and the outflow tube to be provided as a disposable unit of which the pump, in use, is removably located between a base member and a foot pedal of a foot operated operating mechanism.

The invention extends also to a foot operated operating mechanism as such, which is particularly adapted for use with a liquid displacement arrangement as herein envisaged, the operating mechanism comprising

a base member positionable on a floor surface; and

a foot pedal displaceably mounted on the base member,

the base member and the foot pedal each defining a locating formation complementary to one of the locating formations defined respectively by the piston rod and the cylinder of the syringe-type pump of a liquid displacement arrangement with which the operating mechanism is to be used, for permitting the removable location of the said pump between the base member and the foot pedal in a configuration in which displacement of the foot pedal with respect to the base member provides for operation of the pump.

For such a foot operated foot operating mechanism, the foot pedal may be pivotally mounted on the base member and the locating formations may permit relative pivotal movement respectively between the syringe-type pump of a liquid displacement arrangement with which the operating mechanism is to be used and the base member and the said syringe-type pump and the foot pedal, the relative pivotal movement permitting axial displacement of the piston of the said syringe-type pump within its cylinder by the displacement of the foot pedal.

Also for such a foot operated operating mechanism, the foot pedal may be displaceable between a retracted position and a depressed position, which positions coincide respectively with limit positions of the piston within the cylinder of the syringe-type pump of a liquid displacement arrangement with which the operating mechanism is to be used.

In use with a liquid displacement arrangement with which the operating mechanism is to be used, displacement of the pedal of the operating mechanism from its retracted position to its depressed position may provide for displacement of liquid from the cylinder of the syringe-type pump of the said liquid displacement arrangement, whereas displacement of the foot pedal from its depressed position to its retracted position may provide for the displacement of liquid into the said cylinder.

The foot operated mechanism also may include urging means acting between the base member and the foot pedal for urging the foot pedal into its retracted position. Still further, the foot operated operating mechanism may include a stop arrangement that acts between the foot pedal and the base member for determining the fully retracted position and the fully depressed position with respect to the base member.

Particularly in relation to the mode of connection of the inflow tube and the outflow tube to the syringe-type pump of a liquid displacement arrangement as herein envisaged, these tubes either may be permanently connected with respect to the cylinder of the pump, or may be releasably connected with respect thereto.

Further features of the invention, including the mode of use thereof and the benefits associated with the use thereof, are described hereafter with reference to an example illustrated by way of the accompanying diagrammatic drawings. In the drawings:

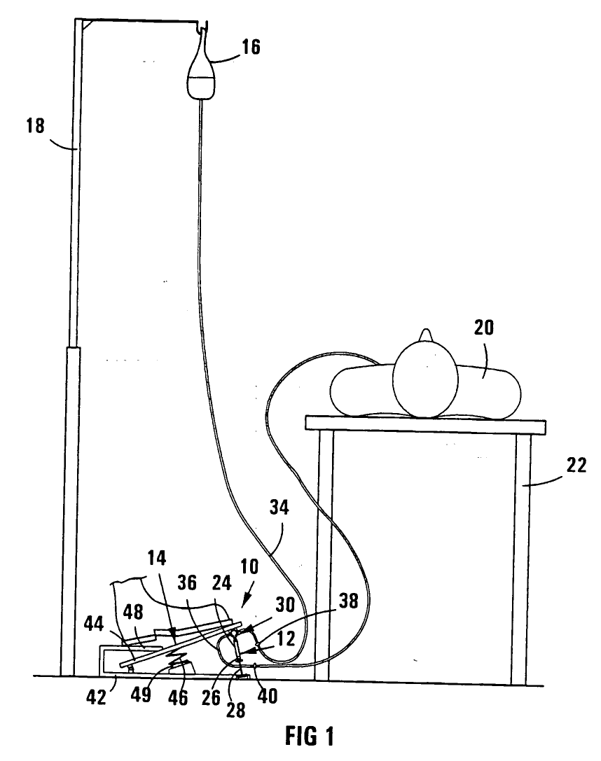

FIG. 1 shows a schematic view of a liquid displacement arrangement, in accordance with the invention; and

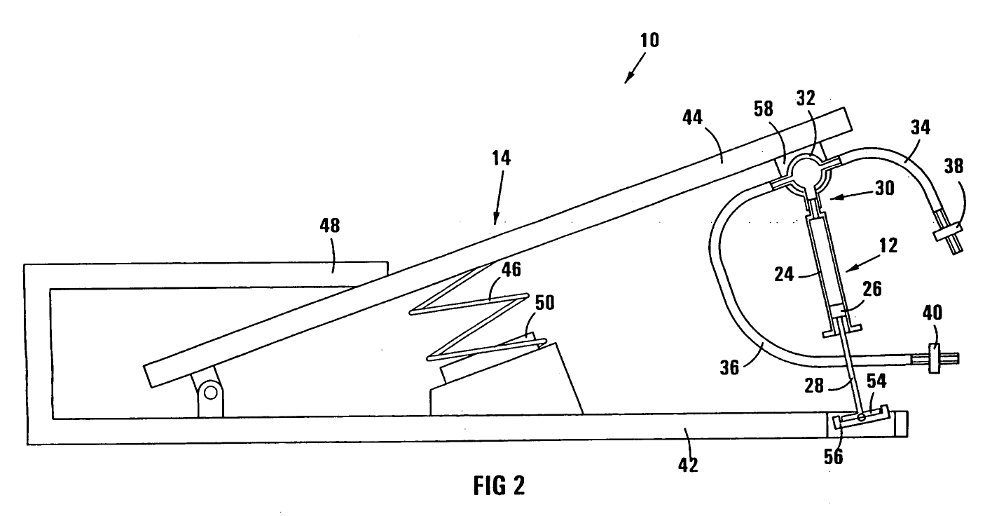

FIG. 2 shows a detailed schematic side view of the main part of the liquid displacement arrangement of FIG. 1.

Referring to the drawings, a liquid displacement arrangement, in accordance with the this invention, is designated generally by the reference numeral 10. The arrangement 10 includes a liquid displacement means, generally designated by the reference numeral 12, and an operating mechanism, generally designated by the numeral 14, for operating the liquid displacement means 12.

The liquid displacement arrangement is adapted particularly for displacing a liquid in a controlled manner to the body of a person, typically from a liquid supply sachet 16 which is suspended at an elevated level as shown by a support stand 18 which permits the level of the sachet 16 to be adjusted, as is described in more detail hereafter. FIG. 1 illustrates also the typical location of the body 20 of a patient lying on a bed structure 22 and requiring a medical procedure that will involve the feed of liquid from the sachet 16 to his body.

The liquid displacement means 12 particularly comprises a syringe-type pump including a cylinder 24, a piston 26 displaceable within the cylinder and a piston rod 28 extending from the piston 26 and projecting from the cylinder 24, the piston rod permitting displacement of the piston 26 within the cylinder 24. The end 30 of the cylinder 24 terminates in a connector formation 32 that it is in liquid communication with the remainder of the cylinder 24, the connector formation 32 having an inflow tube 34 and an outflow tube 36 connected in liquid communication therewith, as shown.

The inflow tube 34, in use of the liquid displacement arrangement 10, is connected to the liquid supply sachet 16 as shown and has a unidirectional valve 38 located in line therewith which permits only flow from the sachet 16 to the cylinder 24. The outflow tube 36 also has a unidirectional valve 40 located in line therewith, the valve 40 permitting flow only from the cylinder 24 to the body 20 of a patient, typically via a liquid feed means (not shown) to which the free end of the outflow tube 36 is connected and which permits required feed of liquid to the body 20.

The operating mechanism 14 includes a base member 42 which has a foot pedal 44 pivotally mounted thereon. A spring 46 acts between the base member and the foot pedal for urging the pedal into its fully retracted position as shown.

Furthermore, a stop arrangement, including a first stop formation 48 and a second stop formation 50, is provided to define respectively the fully retracted position and the fully depressed position of the pedal 44 with respect to the base member 42, these positions coinciding essentially with the limit position of the piston 26 in the cylinder 24 of the syringe-type pump.

As is shown in detail in FIG. 2 of the drawings, the end of the piston rod 28 of the liquid displacement means 12 has a locating formation 54 that cooperates with a complementary locating formation 56 that is pivotally located with respect to the base member 42, providing for releasable pivotal location of this end of the piston rod 28 with respect to the base member 42. The connector formation 32 located at the end 30 of the cylinder 24 itself defines a locating formation that is pivotally located within a complementary locating formation 58 provided near the free end of the foot pedal 44, particularly in the configuration as shown and thereby permitting also relative pivotal displacement between the cylinder 24 and the foot pedal 44. Being releasable from the base member 42 and the foot pedal 44, the liquid displacement means 12 is easily replaceable, whereas the pivotal displacement as described permits the displacement of the piston 26 within the cylinder 24 by the displacement of the foot pedal 44 with respect to the base member 42, without undue sideways forces acting on the piston.

It will be understood that the liquid displacement arrangement 10 will permit, through foot operation, the displacement of liquid from the sachet 16 to the body 20 of a patient, displacement of the foot pedal 44 from its position as shown towards a depressed position providing for displacement of liquid from the cylinder 24 to the body 20 of the patient, whereas reverse displacement of the foot pedal 44 will provide for displacement of further liquid from the sachet 16 into the cylinder 24.

The unidirectional valves 38 and 40 particularly may be configured also to permit a permanent flow of liquid through the flow line defined, with the flow rate being determined by the hydrostatic pressure acting, on the valves, which in turn will be determined by the level at which the sachet 16 is suspended. By permitting adjustability of this level, different continuous flow rates can be provided for and particularly by suspending the sachet 16 at a level beneath, the body 20 of a patient, continuous flow will cease.

It is envisaged that the syringe-type pump may be of a conventional syringe-type construction, whereas the inflow tube 34 and the outflow tube 36 may be of polyvinyl chloride, latex rubber, or the like. It is envisaged in this regard that the entire liquid displacement means 12 will be disposable, the releasability of this means from the operating mechanism 14 facilitating disposal and the use of a fresh liquid displacement means in association with each different patient and/or in association with each different liquid utilized.

The Applicant believes that the liquid displacement arrangement of the invention is particularly suitable for use in procedures associated with uretroscopy and with the manipulation or destruction of ureteric and kidney stones, which involves an elongate scope to enter the ureteric passage. Liquid can then be displaced into the passage for releasing stones, which will in turn permit gripping of stones to be retracted from the said passage. Insofar as the person performing the procedure himself can now operate the liquid displacement arrangement and, particularly, the rate of liquid displacement, control over liquid displacement is facilitated and known problems associated with these procedures alleviated. The stop formation 48 serves in this regard also to optimize the position of the foot of a person, performing a procedure, with respect to the foot pedal 44 (see FIG. 1), i.e. the foot position that will provide maximum control over the operation of the syringe-type pump during the performance of a procedure.

By providing a spring (not shown) to act on the piston rod of the liquid feed means 12 for urging the rod into its extended position as shown, the liquid feed means 12 also can be manually used when separated from the operating mechanism 14, whenever this may be required.

The operating mechanism 14 itself may be made of various different materials, e.g. stainless steel, with the locating formations 56 and 58 thereof being adapted particularly to accommodate the liquid feed means 12, in the configuration described.

1. A liquid displacement arrangement for displacing a liquid in a controlled manner to the body of a person, which comprises
a syringe-type pump including a cylinder, a piston displaceable within the cylinder and a piston rod extending from the piston and projecting from the cylinder to enable displacement of the piston and thereby operation of the pump;
an inflow tube of which one end is connected in liquid communication with the cylinder and the opposite free end is connectable to a liquid supply, the inflow tube having a unidirectional valve located in line therewith that permits only flow towards the cylinder, during operation of the pump;
an outflow tube of which one end is connected in liquid communication with the cylinder and the opposite free end is connectable to a liquid feed means for feeding a liquid to the body of a person, the outflow tube having a unidirectional valve located in line therewith that permits only flow from the cylinder, during operation of the pump,
and in which the piston rod and the cylinder of the syringe-type pump each define a locating formation for removably locating the pump between a base member and a foot pedal of a foot operated operating mechanism in a configuration in which displacement of the foot pedal provides for required displacement of the piston of the pump for the operation of the pump.

2. A liquid displacement arrangement as claimed in claim 1, which includes a connector formation which communicates with the cylinder of the syringe-type pump and to which the inflow tube and the outflow tube are connected for providing their required liquid communication with the cylinder.

3. A liquid displacement arrangement as claimed in claim 1 or claim 2, in which the free end of the inflow tube is connectable to a liquid supply container and permits the container to be positioned at different height levels with respect to the body of a person to which a liquid from the liquid supply container must be displaced, in use of the liquid displacement arrangement, and in which the unidirectional valves located in line with the inflow tube and the outflow tube respectively, permit liquid flow in the permitted direction when the hydrostatic pressure acting on the valves, being determined by the position of a liquid supply container to which the inflow tube is connected, in use, exceeds a predetermined pressure level.

4. A liquid displacement arrangement as claimed in any one of claims 1 to 3, which includes a foot operated operating mechanism for operating the syringe-type pump, the operating mechanism comprising
a base member positionable on a floor surface; and
a foot pedal displaceably mounted on the base member,
the base member and the foot pedal each defining a locating formation complementary to one of the locating formations defined respectively by the piston rod and the cylinder of the syringe-type pump, permitting the removable location of the pump between the base member and the foot pedal in a configuration in which displacement of the foot pedal with respect to the base member provides for operation of the pump.

5. A liquid displacement arrangement as claimed in claim 4, in which the locating formations defined respectively by the piston rod and the cylinder of the syringe-type pump are located at the operative remote ends of the piston rod and the cylinder.

6. A liquid displacement arrangement as claimed in claim 4 or claim 5, in which the foot pedal is pivotally mounted on the base member and the locating formations permit relative pivotal movement respectively between the syringe-type pump and the base member and the syringe-type pump and the foot pedal, the relative pivotal movement permitting axial displacement of the piston of the syringe-type pump by the displacement of the foot pedal.

7. A liquid displacement arrangement as claimed in any one of claims 4 to 6, in which the foot pedal is displaceable between a retracted position and a depressed position, which positions coincide respectively with limit positions of the piston of the syringe-type pump within its cylinder.

8. A liquid displacement arrangement as claimed in claim 7, in which, in use of the liquid displacement arrangement, displacement of the pedal from its retracted position to its depressed position provides for displacement of liquid from the cylinder of the syringe-type pump, whereas displacement of the foot pedal from its depressed position to its retracted position provides for displacement of liquid into the cylinder.

9. A liquid displacement arrangement as claimed in claim 8, in which the foot operated operating mechanism includes urging means acting between the base member and the foot pedal for urging the foot pedal into its retracted position.

10. A liquid displacement arrangement as claimed in any one of claims 7 to 9, in which the foot operated foot operating mechanism includes a stop arrangement that acts between the foot pedal and the base member for determining the fully retracted position and the fully depressed position of the pedal with respect to the base member.

11. A liquid displacement arrangement as claimed in any one of claims 7 to 10, in which the syringe-type pump includes urging means acting on the piston rod for urging the piston rod into its fully extended position with respect to the cylinder.

12. A liquid displacement arrangement as claimed in any one of the preceding claims, in which the syringe-type pump, the inflow tube and the outflow tube are provided as a disposable unit of which the pump is removably locatable between a base member and a foot pedal of a foot operated operating mechanism.

13. A foot operated operating mechanism for use with a liquid displacement arrangement as claimed in any one of claims 1 to 3, which mechanism comprises
a base member positionable on a floor surface; and
a foot pedal displaceably mounted on the base member,
the base member and the foot pedal each defining a locating formation complementary to one of the locating formations defined respectively by the piston rod and the cylinder of the syringe-type pump of a liquid displacement arrangement with which the operating mechanism is to be used, for permitting the removable location of the said pump between the base member and the foot pedal in a configuration in which displacement of the foot pedal with respect to the base member provides for operation of the pump.

14. A foot operated operating mechanism as claimed in claim 13, in which the foot pedal is pivotally mounted on the base member and the locating formations permit relative pivotal movement respectively between the syringe-type pump of a liquid displacement arrangement with which the operating mechanism is to be used and the base member and the said syringe-type pump and the foot pedal, the relative pivotal movement permitting axial displacement of the piston of the said syringe-type pump within its cylinder by the displacement of the foot pedal.

15. A foot operated operating mechanism as claimed in claim 13 or claim 14, in which the foot pedal is displaceable between a retracted position and a depressed position, which positions coincide respectively with limit positions of the piston within the cylinder of the syringe-type pump of a liquid displacement arrangement with which the operating mechanism is to be used.

16. A foot operated operating mechanism as claimed in claim 15, in which, in use with a liquid displacement arrangement with which the operating mechanism is to be used, displacement of the pedal from its retracted position to its depressed position provides for displacement of liquid from the cylinder of the syringe-type pump of the said liquid displacement arrangement, whereas displacement of the foot pedal from its depressed position to its retracted position provides for the displacement of liquid into the said cylinder.

17. A foot operated operating mechanism as claimed in claim 16, which includes urging means acting between the base member and the foot pedal for urging the foot pedal into its retracted position.

18. A foot operated operating mechanism as claimed in any one of claims 15 to 17, which includes a stop arrangement that acts between the foot pedal and the base member for determining the fully retracted position and the fully depressed position of the pedal with respect to the base member.

19. A liquid displacement arrangement for displacing a liquid in a controlled manner to the body of a person substantially as described in the specification with reference to and as illustrated in the accompanying drawings.

20. A foot operated operating mechanism substantially as described in the specification with reference to and as illustrated in the accompanying drawings.
